(19) United States
(12) Patent Application Publication
(10) Pub. No.: US 2010/0099773 A1
(43) Pub. Date: Apr. 22, 2010

(54) INCLUSION COMPLEX OF SIBUTRAMINE AND BETA-CYCLODEXTRIN

(76) Inventors: Jae-Sun Kim, Gyeonggi-do (KR); Nam Ho Kim, Gyeonggi-do (KR); Jin Young Lee, Gyeonggi-do (KR); Nam Kyu Lee, Gyeonggi-do (KR); Joon Gyo Oh, Gyeonggi-do (KR); Yong Youn Hwang, Gyeonggi-do (KR); Dong-Chul Shin, Gyeonggi-do (KR); Yoon-Jung Lee, Gyeonggi-do (KR); Key An Um, Gyeonggi-do (KR); Sun Ho Kim, Seoul (KR); Jin-Heung Sung, Gyeonggi-do (KR)

Correspondence Address: FROMMER LAWRENCE & HAUG, 745 FIFTH AVENUE- 10TH FL., NEW YORK, NY, 10151, US

(21) Appl. No.: 12/444,080

(22) PCT Filed: Nov. 22, 2007
(86) PCT No.: PCT/KR2007/005922

§ 371 Date: Apr. 2, 2009

(30) Foreign Application Priority Data
Nov. 22, 2006 (KR) 10-2006-0115916
Nov. 22, 2007 (KR) 10-2007-0119483

(51) Int. Cl. A61K 31/135; A61P 25/00

(52) U.S. Cl. 514/646

(57) ABSTRACT

The present invention relates to a sibutramine-containing inclusion complex having superior storage stability, and particularly to a pharmaceutically stable inclusion complex suitable for the drug formulation, which prepared by reacting a sibutramine (N,N-dimethyl-1-[1-(4-chlorophenyl)-cyclobutyl]-3-methylbutylamine) of Formula 1 and beta-cyclodextrin in a predetermined ratio, its preparation method and a pharmaceutical composition comprising the same.

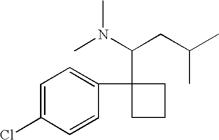

### TECHNICAL FIELD

The present invention relates to a sibutramine-containing inclusion complex having superior storage stability, the preparation method and the use thereof.

### BACKGROUND ART

Sibutramine has been known effective for the prevention and treatment of hypochondria, Parkinson s disease and obesity [English patent No. 2,098,602; Korean patent publication No. 90-00274; WO 88/06444; and Korean patent publication No. 99-164435].

Further, sibutramine may be used to decrease insulin tolerance or improve glucose tolerance, and is known useful for the prevention and treatment of diseases such as gout, hyperuricaemia, hyperlipidemia, osteoarthritis, anxiety disorders, somnipathy, sexual dysfunction, chronic fatigue syndrome and cholelithiasis [U.S. Pat. Nos. 6,174,925; 5,459,164; 6,187,820; 6,162,831; 6,232,347; 6,355,685; 6,365,631; 6,376,554; 6,376,551; and 6,376,552].

However, sibutramine exists in an oily state, and thus it is difficult to handle it for pharmaceutical use. For the manufacture of a pharmaceutical composition suitable to administration, it is essential that sibutramine be changed into a pharmaceutically acceptable acid salt before use.

English patent No. 2,098,602 and Korean patent publication No. 90-274 disclose processes of preparing anhydrous sibutramine hydrochloride as a pharmaceutically acceptable acid salt of sibutramine. However, the anhydrous sibutramine hydrochloride is has a relatively high hygroscopic property, and it is difficult to maintain a constant content of sibutramine in a pharmaceutical composition. Absorbed water may cause hydrolysis or chemical decomposition of the active ingredient (sibutramine), thereby drastically decreasing the efficacy of sibutramine. For this reason, it is difficult to use anhydrous sibutramine hydrochloride as an active ingredient of a pharmaceutical composition.

To overcome the aforementioned problems, English patent No. 2,184,122 and Korean patent publication No. 94-8913 disclose a process of preparing non-hygroscopic sibutramine hydrochloride monohydrate of Formula 2.

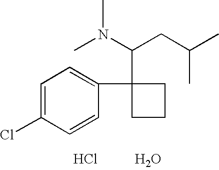

Sibutramine hydrochloride monohydrate dose not have the problem shown in anhydrous sibutramine hydrochloride caused by its hygroscopic property. Therefore, by the development in the utilization of sibutramine hydrochloride monohydrate, sibutramine began to be used in preparing a therapeutic agent. More specifically, sibutramine hydrochloride monohydrate has been used as an active ingredient of Meridia or Reductil™, a therapeutic drug for the treatment of obesity.

### DISCLOSURE OF INVENTION

### Technical Problem

To overcome the aforementioned problems of sibutramine relating to hygroscopic property and stability, the present inventors have exerted extensive researches. As a result, the present invention has been completed on a basis of the findings that an inclusion complex prepared by reacting sibutramine and beta-cyclodextrin in a predetermined molar ratio is superior to an acid salt or a free base in terms of storage stability and render properties suitable for the manufacture of drug formulation.

Therefore, the present invention aims to provide a sibutramine inclusion complex having superior storage stability, and a method of its preparation and the use thereof.

### Technical Solution

The present invention relates to a sibutramine inclusion complex having superior storage stability, which comprises sibutramine of Formula 1 and beta-cyclodextrin.

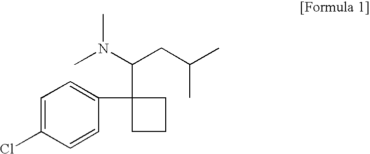

Further, the present invention relates to a process of preparing a sibutramine inclusion complex, which comprises:

1) obtaining a sibutramine-containing solution by dissolving sibutramine and beta-cyclodextrin in an acidic solution;

2) obtaining a solution containing sibutramine and beta-cyclodextrin by adding beta-cyclodextrin in the sibutramine-containing acidic solution, and stirring the solution containing sibutramine and beta-cyclodextrin at 20-60° C.;

3) neutralizing the mixed solution by adding a base; and

4) cooling the neutralized solution to 0-40° C., followed by filtration, washing and drying.

The present invention also relates to a pharmaceutical composition for the treatment and prevention of hypochondria and obesity, which comprises an inclusion complex herein as an active ingredient.

### Advantageous Effects

Due to the superior storage stability of the material, a sibutramine inclusion complex according to the present invention may be stably stored for a long period of time, and easily prepared into a drug formulation. The inclusion complex herein is also resistant to temperature and humidity during the manufacturing process without being decomposed. Further, the inclusion complex has dissolution rate superior to sibutramine per se, and the drug formulation of the inclusion complex has comparable dissolution rate to that of commercially available drugs.

### BRIEF DESCRIPTION OF THE DRAWINGS

FIGS. 1 and 2 are the powder X-ray diffraction spectra of sibutramine inclusion complex prepared according to the present invention.

FIG. 3 is a graph comparing the dissolution rates of a capsule comprising an inclusion complex herein and a commercially available Reductil™ capsule.

### MODE FOR THE INVENTION

Hereunder is provided a detailed description of the present invention.

The present invention relates to a pharmaceutically stable inclusion complex suitable for a drug formulation, which is prepared by reacting sibutramine (N,N-dimethyl-1-[1-(4-chlorophenyl)-cyclobutyl]-3-methylbutylamine) of Formula 1 and beta-cyclodextrin in a predetermined ratio, the preparation method thereof and a pharmaceutical composition for the treatment and prevention of hypochondria and obesity, which comprises an inclusion complex herein as an active ingredient.

An inclusion complex herein is not a simple mixture of the ingredients, but has a structure where sibutramine molecules are chemically bound to beta-cyclodextrin molecules. An inclusion complex is superior to the conventional acid salt of sibutramine or a sibutramine free base in storage stability.

Hereunder is provided a detailed description of a sibutramine inclusion complex herein.

Sibutramine used in the present invention refers to a sibutramine base or a sibutramine salt. Preferable examples of the sibutramine salt include hydrochloride, methane sulfonate, ethane sulfonate, benzene sulfonate, camphorsulfonate, tartrate, maleate, malate, mandelate, salicylate and isethionate.

In step 1), sibutramine is dissolved in an acidic solution. Organic or inorganic acid solution may be used as the acidic solution. Preferable examples of the acid include hydrochloric acid, sulfuric acid, phosphoric acid and acetic acid.

In step 2), beta-cyclodextrin is added to the sibutramine acidic solution, followed stirring at an elevated temperature. The stirring is preferred to be conducted at 20-60° C., more preferably 30-40° C. When the temperature is lower than 20° C., the amount of solvent required for dissolving cyclodextrin may increase and the inclusion efficiency may decrease. When the temperature is higher than 60° C., drugs may be decomposed.

The solution may further comprise at least one water-soluble polymer selected from the group consisting of polyethyleneglycol (PEG), polyvinylpyrrolidone (PVP), carboxymethyl cellulose (CMC), hydroxypropyl cellulose (HPC), hydroxymethyl cellulose (HMC), hydroxyethyl cellulose (HEC), hydroxypropylmethyl cellulose (HPMC) and hydroxypropylethyl cellulose (HPEC).

Beta-cyclodextrin derivatives may also be used as the beta-cyclodextrin in the present invention. Preferable example is β-cyclodextrins or their derivatives comprising pores with a diameter of 6.0-6.5 Å. Beta-cyclodextrin is preferred to be used in the amount of 0.5-4 equivalents, more preferably 1.0-4 equivalents, most preferably 1.5-3 equivalents relative to one equivalent of sibutramine. When the content of beta-cyclodextrin is higher than the aforementioned upper range, the content of inclusion complex may decrease due to a large amount of non-reacted cyclodextrin. When the content is less than the aforementioned lower limit, sufficient stability may not be achieved.

In step 3), the solution is neutralized by the addition of a base. Examples of the base include alkali metal hydroxide such as sodium hydroxide, potassium hydroxide, barium hydroxide and calcium hydroxide. The solution is neutralized at 0-50° C., preferably 0-25° C. When the temperature is lower than 0° C., other impurities or non-included cyclodextrin may also be precipitated due to overcooling. When the temperature is higher than 50° C., the production of impurities may increase.

In step 4), the solution is cooled, filtered, washed and dried, thereby producing an inclusion complex. The cooling is conducted at 0-40° C., preferably 0-25° C. When the temperature is lower than 0° C., other impurities or non-included cyclodextrin may also be precipitated due to overcooling. When the temperature is higher than 40° C., the yield may drastically decrease. Further, inclusion complex may be finally obtained by washing the filtrate with a small amount of cold water several times and drying the washed filtrate.

It is ascertained in the present invention that thereby obtained inclusion complex may be stably stored for a long period of time, and easily prepared into a drug formulation due to the superior storage stability of the material per se. An inclusion complex herein is also resistant to temperature and humidity during the manufacturing process.

To find a material superior to the known salts of sibutramine, the present inventors have exerted extensive researches relating to various inclusion by using beta-cyclodextrin.

As a result, the present inventors have ascertained that an inclusion complex, which is prepared only when appropriate conditions are satisfied and maintained, is a pharmaceutically useful novel form of sibutramine superior in physicochemical properties, although it is not of a salt form.

This is opposite to the conventional result in that a sibutramine base is oily liquid and may form a stable salt by an extremely limited acid salt. This result ascertains that a composition suitable for the preparation of medical formulation may achieved by the inclusion reaction without using a salt. Further, an inclusion complex is prepared by the inclusion of sibutramine base or salt, and does not comprise an acid salt. An inclusion complex also has a similar crystalline form and an unexpectedly superior stability, and is suitable for the preparation of medical formulation of sibutramine. Further, a pharmaceutical composition for the treatment and prevention of hypochondria and obesity, which comprises an inclusion complex of the present invention as an active ingredient, may be prepared as described below.

A medicine for oral administration may be prepared by mixing the inclusion complex with pharmaceutically acceptable carriers such as an excipient, a binding agent, a disintegrant, a lubricant and a sweetening agent. Preferable examples of the excipient include microcrystalline cellulose and lactose. Preferable examples of the binding agent include povidone and hydroxypropyl cellulose. Preferable examples of the disintegrant include croscarmellose sodium, sodium starch glycolate and calcium carboxymethyl cellulose. Preferable examples of the lubricant include colloidal silica dioxide, magnesium stearate and talc. Further, examples of the dosage form of the medicine for oral administration include tablets, capsules, liquids, suspensions and granules. Although the effective dose of sibutramine varies with the age of a patient or seriousness of disease, 20-200 mg, preferably 40-150 mg of sibutramine may be daily administered on the basis of an inclusion complex herein.

The present invention is described more specifically with reference to the following

Examples. Examples herein are meant only to illustrate the present invention, but they should not be construed as limiting the scope of the claimed invention.

### Example 1

### Preparation of Inclusion Complex Comprising Sibutramine and Beta-Cyclodextrin

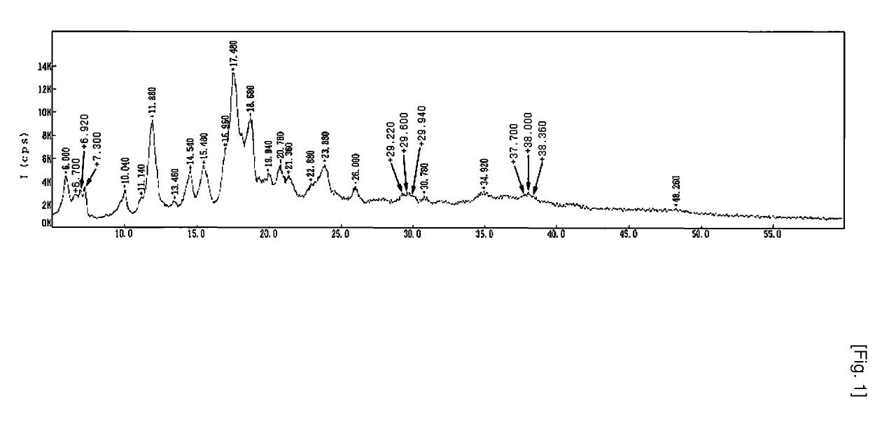

Sibutramine free base (28 g) and distilled water (6 L) were added to a flask, and then 200 mL of 1N-HCl(aq) was added thereto. The mixture was stirred for 20 minutes to completely dissolving the sibutramine free base. Beta-cyclodextrin was added to this solution in the amount of 256 g (2.0 equivalents relative to one equivalent of sibutramine, only relative equivalent is described hereinafter), and the resulting solution was stirred at 35° C. for 30 minutes and stirred further at 25° C. for 2 hours. 1N NaOH(aq) 200 mL was slowly added, and the solution was stirred at 25° C. for 3 hours. Solid precipitates were filtered through a filter paper under reduced pressure, and washed with distilled water. The product was vacuum-dried for 18 hours at 50° C., thereby obtaining a white solid compound (245 g, yield 96%). The crystalline state of thereby obtained sibutramine inclusion complex was analyzed by using an X-ray diffraction (XRD). As a result, it was ascertained that the sibutramine inclusion complex is a crystal having characteristic diffraction angles [FIG. 1].

¹H-NMR (300 MHz, DMSO-d₆) (ppm): 0.84(d, 3H), 0.92(d, 3H), 1.05-1.14(m, 2H), 1.44-1.48(m, 1H), 1.64-1.68(m, 1H), 1.87-1.91(m, 1H), 2.08-2.12(m, 2H), 2.11(s, 6H), 2.14-2.23(m, 1H), 2.36-2.43(m, 1H), 2.86(dd, 1H), 3.21-3.45(m, 72H), 3.47-3.75(m, 40H), 4.46(t, 14H), 4.82(d, 14H), 5.67(d, 14H), 5.71(d, 14H), 7.19(d, 2H), 7.31(d, 2H)

### Example 2

### Preparation of Inclusion Complex of Sibutramine and Beta-Cyclodextrin

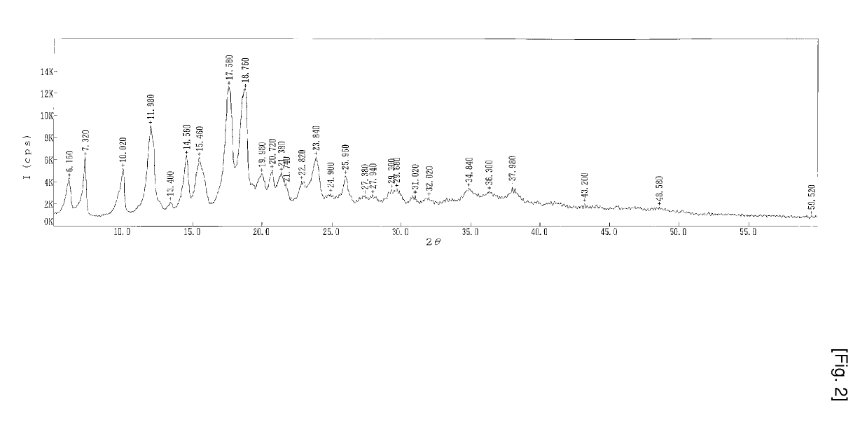

Sibutramine hydrochloride monohydrate (33.4 g) and distilled water (6 L) were added in a flask, and then 1N-HCl(aq) 100 mL was added thereto. The mixture was stirred for 20 minutes to completely dissolving sibutramine hydrochloride monohydrate. Beta-cyclodextrin (256 g, 2.0 equivalents) was added to this solution, and the resulting solution was stirred at 35° C. for 30 minutes and stirred further at 25° C. for 2 hours. 1N NaOH(aq) 200 mL was slowly added, and the solution was stirred at 25° C. for 3 hours. Solid precipitates were filtered through a filter paper under reduced pressure, and washed with distilled water. The product was vacuum-dried for 18 hours at 50° C., thereby obtaining a white solid compound (239 g, yield 94%). The resulting sibutramine inclusion complex was subject to NMR and XRD analyses, and the results are similar to those of Example 1 [FIG. 2].

¹H-NMR (300 MHz, DMSO-d₆) (ppm): 0.84(d, 3H), 0.91(d, 3H), 1.05-1.14(m, 2H), 1.44-1.47(m, 1H), 1.64-1.67(m, 1H), 1.87-1.90(m, 1H), 2.08-2.12(m, 2H), 2.10(s, 6H), 2.14-2.22(m, 1H), 2.36-2.42(m, 1H), 2.85(dd, 1H), 3.21-3.45(m, 40H), 3.49-3.67(m, 56H), 4.44(t, 14H), 4.82(d, 14H), 5.67(d, 14H), 5.71(d, 14H), 7.18(d, 2H), 7.30(d, 2H)

### Example 3

### Preparation of Inclusion Complex of Sibutramine and Beta-Cyclodextrin

Sibutramine free base (2.8 g) and distilled water (600 mL) were added to a flask, and then 1N-HCl(aq) 20 mL was added thereto. The mixture was stirred for 20 minutes to completely dissolving sibutramine free base. Beta-cyclodextrin (6.4 g, 0.5 equivalents) was added to this solution, and the resulting solution was stirred at 35° C. for 30 minutes and stirred further at 25° C. for 2 hours. 1N NaOH(aq) 20 mL was slowly added, and the solution was stirred at 25° C. for 3 hours. Solid precipitates were filtered through a filter paper under reduced pressure, and washed with distilled water. The product was vacuum-dried for 18 hours at 50° C., thereby obtaining a white solid compound (8.1 g, yield 32%).

¹H-NMR (300 MHz, DMSO-d₆) (ppm): 0.84(d, 3H), 0.92(d, 3H), 1.05-1.14(m, 2H), 1.44-1.48(m, 1H), 1.65-1.68(m, 1H), 1.86-1.91(m, 1H), 2.10-2.15(m, 2H), 2.10(s, 6H), 2.14-2.21(m, 1H), 2.34-2.46(m, 1H), 2.87(dd, 1H), 3.20-3.44(m, 72H), 3.47-3.76(m, 40H), 4.45(t, 14H), 4.82(d, 14H), 5.66(d, 14H), 5.71(d, 14H), 7.20(d, 2H), 7.32(d, 2H)

### Example 4

### Preparation of Inclusion Complex of Sibutramine and Beta-Cyclodextrin

A white solid compound was obtained (14.1 g, yield 55%) the same as described in Example 3 except that sibutramine free base (2.8 g) and beta-cyclodextrin (12.8 g, 1.0 equivalent) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 5

### Preparation of Inclusion Complex of Sibutramine and Beta-Cyclodextrin

A white solid compound was obtained (19.4 g, yield 76%) the same as described in Example 3 except that sibutramine free base (2.8 g) and beta-cyclodextrin (19.2 g, 1.5 equivalents) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 6

### Preparation of Inclusion Complex of Sibutramine and Beta-Cyclodextrin

A white solid compound was obtained (24.0 g, yield 94%) the same as described in Example 3 except that sibutramine free base (2.8 g) and beta-cyclodextrin (32.0 g, 2.5 equivalents) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 7

### Preparation of Inclusion Complex of Sibutramine and Beta-Cyclodextrin

A white solid compound was obtained (23.1 g, yield 90.5%) the same as described in Example 3 except that sibutramine free base (2.8 g) and beta-cyclodextrin (38.4 g, 3.0 equivalents) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 8

### Preparation of Inclusion Complex of Sibutramine and Beta-Cyclodextrin

Sibutramine hydrochloride monohydrate (3.3 g) and distilled water (600 mL) were added to a flask, and 1N-HCl(aq) 10 mL was also introduced to the flask. The mixture was stirred for 20 minutes to completely dissolving sibutramine hydrochloride monohydrate. Beta-cyclodextrin (6.4 g, 0.5 equivalents) was added to this solution, and the resulting solution was stirred at 35° C. for 30 minutes and stirred further at 25° C. for 2 hours. 1N NaOH(aq) 20 mL was slowly added, and the solution was stirred at 25° C. for 3 hours. Solid precipitates were filtered through a filter paper under reduced pressure, and washed with distilled water. The product was vacuum-dried for 18 hours at 50° C., thereby obtaining a white solid compound (7.9 g, yield 31%).

¹H-NMR (300 MHz, DMSO-d₆) (ppm): 0.84(d, 3H), 0.92(d, 3H), 1.05-1.14(m, 2H), 1.44-1.48(m, 1H), 1.65-1.68(m, 1H), 1.86-1.91(m, 1H), 2.10-2.15(m, 2H), 2.10(s, 6H), 2.14-2.21(m, 1H), 2.34-2.46(m, 1H), 2.87(dd, 1H), 3.20-3.44(m, 72H), 3.47-3.76(m, 40H), 4.45(t, 14H), 4.82(d, 14H), 5.66(d, 14H), 5.71(d, 14H), 7.20(d, 2H), 7.32(d, 2H)

### Example 9

### Preparation of Inclusion Complex of Sibutramine and Beta-Cyclodextrin

A white solid compound was obtained (14.3 g, yield 56%) the same as described in Example 8 except that sibutramine hydrochloride monohydrate (3.3 g) and beta-cyclodextrin (12.8 g, 1.0 equivalent) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 10

### Preparation of Inclusion Complex of Sibutramine and Beta-Cyclodextrin

A white solid compound was obtained (18.8 g, yield 74%) the same as described in

Example 8 except that sibutramine hydrochloride monohydrate (3.3 g) and beta-cyclodextrin (19.2 g, 1.5 equivalents) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 11

### Preparation of Inclusion Complex of Sibutramine and Beta-Cyclodextrin

A white solid compound was obtained (23.2 g, yield 91%) the same as described in Example 8 except that sibutramine hydrochloride monohydrate (3.3 g) and beta-cyclodextrin (32.0 g, 2.5 equivalents) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Example 12

### Preparation of Inclusion Complex of Sibutramine and Beta-Cyclodextrin

A white solid compound was obtained (24.5 g, yield 96%) the same as described in Example 8 except that sibutramine hydrochloride monohydrate (3.3 g) and beta-cyclodextrin (38.4 g, 3.0 equivalents) were used.

¹H-NMR (300 MHz, DMSO-d₆) (ppm): 0.84(d, 3H), 0.92(d, 3H), 1.06-1.14(m, 2H), 1.43-1.48(m, 1H), 1.64-1.67(m, 1H), 1.87-1.92(m, 1H), 2.08-2.11(m, 2H), 2.11(s, 6H), 2.15-2.23(m, 1H), 2.37-2.43(m, 1H), 2.88(dd, 1H), 3.21-3.47(m, 72H), 3.47-3.77(m, 40H), 4.48(t, 14H), 4.82(d, 14H), 5.69(d, 14H), 5.71(d, 14H), 7.18(d, 2H), 7.30(d, 2H)

### Experimental Example 1

### Test for Storage Stability

Solution Stability (pH 5.2)

Sibutramine free base, sibutramine hydrochloride monohydrate and sibutramine inclusion complex (Examples 1 and 2) were compared in terms of solution stability at high temperature.

Specifically, each compound was dissolved into the concentration of 1 mg/mL and pH 5.2, and the solution was moved to a 20 mL vial. Solution stability test was conducted at 60° C. and 70° C., respectively, by measuring the content of impurities with high performance liquid chromatography (HPLC) after the storage for 4, 7 and 14 days. Tables 1 and 2 show the increase in the content of impurities.

TABLE 1
Solution stability test at 60 C. (Total impurities, %)

| Compound | Initial | 4^{th} day | 7^{th} day | 14^{th} day |
|---|---|---|---|---|
| Sibutramine free base | 0.00 | 7.59 | 13.77 | 17.16 |
| Sibutramine hydrochloride | 0.00 | 0.02 | 0.02 | 0.09 |
| monohydrate | | | | |
| Inclusion complex of Example 1 | 0.00 | 0.02 | 0.01 | 0.05 |
| Inclusion complex of Example 2 | 0.00 | 0.02 | 0.02 | 0.04 |

TABLE 2
Solution stability test at 70 C. (Total impurities, %)

| Compound | Initial | 4^{th} day | 7^{th} day | 14^{th} day |
|---|---|---|---|---|
| Sibutramine free base | 0.00 | 10.04 | 17.05 | 20.52 |
| Sibutramine hydrochloride | 0.00 | 1.56 | 2.09 | 4.49 |
| monohydrate | | | | |
| Inclusion complex of Example 1 | 0.00 | 0.04 | 0.07 | 0.25 |
| Inclusion complex of Example 2 | 0.00 | 0.03 | 0.08 | 0.27 |

As shown in Tables 1 and 2, a less amount of impurities was produced by the sibutramine inclusion complex than by the sibutramine free base or sibutramine hydrochloride monohydrate, thus ascertaining the superior stability of the sibutramine inclusion complex. That is, the sibutramine inclusion complex of the present invention showed the improvement in storage stability compared to that of sibutramine free base or sibutramine hydrochloride monohydrate.

Temperature & Humidity Stability

Sibutramine free base, sibutramine hydrochloride monohydrate and sibutramine inclusion complex (Examples 1 and 2) were compared in terms of temperature and humidity stability.

Specifically, each compound was stored under the condition selected from the group consisting of 60° C. 75%, 60° C. 93% and 70° C. 75% for 2 weeks. The content impurities was measured with high performance liquid chromatography (HPLC). Table 3 shows the increase in the content of impurities.

TABLE 3

| | | 60° C., | | 70° C., |
|---|---|---|---|---|
| Compound | Initial | 75% | 60° C., 93% | 75% |
| Sibutramine free base | 0.00 | 5.65 | 6.09 | 8.27 |
| Sibutramine hydrochloride | 0.00 | 0.06 | 0.07 | 0.13 |
| monohydrate | | | | |
| Inclusion complex of Example 1 | 0.00 | 0.00 | 0.00 | 0.00 |
| Inclusion complex of Example 2 | 0.00 | 0.00 | 0.00 | 0.01 |
| Mixture of sibutramine and | 0.00 | 0.17 | 0.40 | 1.64 |
| beta-cyclodextrin (1:2) | | | | |

Table 3 shows that the free base is most unstable and the sibutramine inclusion complex is most stable. The inclusion complex produces a less amount of impurities than the mixture of sibutramine and beta-cyclodextrin, which ascertains that the inclusion complex of Example 1 or 2 is different from a simple mixture of sibutramine and beta-cyclodextrin.

3) Photo-Stability

Sibutramine free base, sibutramine hydrochloride monohydrate and sibutramine inclusion complex (Examples 1 and 2) were compared in terms of photo-stability.

After UV-irradiation for 120 hours (total radiation dosage: 200 watt), the content of impurities was measured with high performance liquid chromatography (HPLC). Table 4 shows the increase in the content of impurities.

TABLE 4

| Compound | Initial | UV |
|---|---|---|
| Sibutramine free base | 0.00 | 0.33 |
| Sibutramine hydrochloride monohydrate | 0.06 | 0.11 |
| Inclusion complex of Example 1 | 0.00 | 0.05 |
| Inclusion complex of Example 2 | 0.00 | 0.06 |

As shown in Table 4, the sibutramine inclusion complex produces a less amount of impurities compared than sibutramine free base or sibutramine hydrochloride monohydrate, thereby ascertaining the photo-stability of the sibutramine inclusion complex.

### Example 13

### Preparation of Capsule by Using Inclusion Complex

An inclusion complex of Example 1 (81 mg) was mixed with microcrystalline cellulose (95 mg) and sodium stearyl fumarate (4 mg). The mixture was filled in a No. 5 gelatin capsule by using an appropriate device.

### Experimental Example 2

### Dissolution Effect

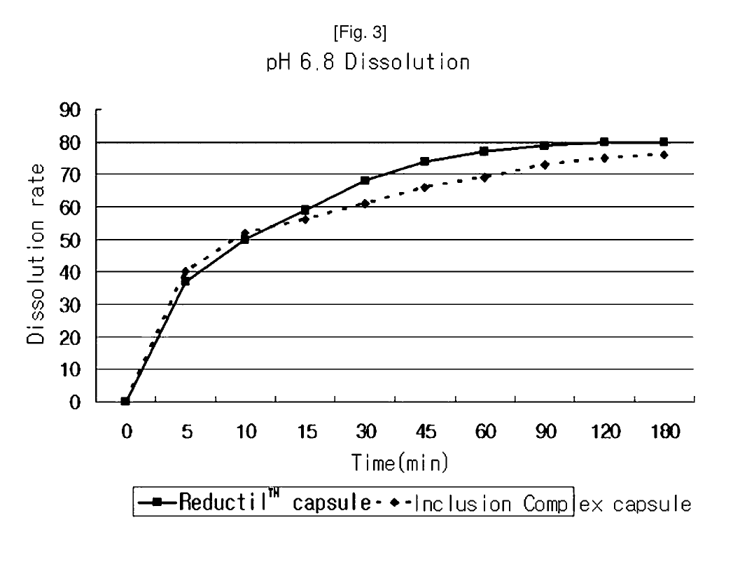

The capsule of Example 13 was compared with a commercially available Reductil™ capsule in terms of dissolution rate in a simulated intestinal fluid (pH 6.8) under the condition of 37° C. and 50 rpm. The results are presented in Table 5 and FIG. 3.

TABLE 5

| | | Dissolution time (minutes) | | | | | | | | | |
|---|---|---|---|---|---|---|---|---|---|---|---|
| Capsule | | 0 | 5 | 10 | 15 | 30 | 45 | 60 | 90 | 120 | 180 |
| Dissolution rate | Reductil ™ capsule | 0 | 37 | 50 | 59 | 68 | 74 | 77 | 79 | 80 | 80 |
| (%) | Example 13 | 0 | 40 | 52 | 56 | 61 | 66 | 69 | 73 | 75 | 76 |

1. An inclusion complex comprising sibutramine and beta-cyclodextrin.

2. The inclusion complex of claim 1, which comprises 0.5-4 equivalents of the beta-cyclodextrin relative to one equivalent of sibutramine.

3. The inclusion complex of claim 2, which comprises 1.0-4 equivalents of the beta-cyclodextrin relative to one equivalent of sibutramine.

4. The inclusion complex of claim 3, which comprises 1.5-3.0 equivalents of the beta-cyclodextrin relative to one equivalent of sibutramine.

5. A process of preparing a sibutramine-containing inclusion complex, which comprises:
1) obtaining a sibutramine-containing solution by dissolving sibutramine and beta-cyclodextrin in an acidic solution;
2) obtaining a solution containing sibutramine and beta-cyclodextrin by adding beta-cyclodextrin in the sibutramine-containing acidic solution, and stirring the solution containing sibutramine and beta-cyclodextrin at 20-60° C.;
3) neutralizing the mixed solution by adding a base; and
4) cooling the neutralized solution to 0-40° C., followed by filtration, washing and drying.

6. The process of claim 5, wherein the acidic solution used in the step 1) is a solution of an acid selected from the group consisting of hydrochloric acid, sulfuric acid, phosphoric acid and acetic acid.

7. The process of claim 6, wherein the acidic solution is hydrochloric acid.

8. The process of claim 5, wherein the stirring in the step 2) is conducted at 30-40° C.

9. The process of claim 5, wherein the base used in the step 3) is an alkali metal hydroxide.

10. The process of claim 9, wherein the alkali metal hydroxide is selected from the group consisting of sodium hydroxide, potassium hydroxide, barium hydroxide and calcium hydroxide.

11. The process of claim 10, wherein the alkali metal hydroxide is sodium hydroxide.

12. The process of claim 5, wherein the cooling in the step 4) is conducted at 0-25° C.

13. A composition for the treatment and prevention of hypochondria and obesity, which comprises the inclusion complex of claim 1 as an active ingredient.

14. A composition for the treatment and prevention of hypochondria and obesity, which comprises the inclusion complex of claim 2 as an active ingredient.

15. A composition for the treatment and prevention of hypochondria and obesity, which comprises the inclusion complex of claim 3 as an active ingredient.

16. A composition for the treatment and prevention of hypochondria and obesity, which comprises the inclusion complex of claim 4 as an active ingredient.
